(19) United States
(12) Patent Application Publication
(10) Pub. No.: US 2005/0147980 A1
(43) Pub. Date: Jul. 7, 2005

(54) Nucleic acid sequencing by Raman monitoring of uptake of nucleotides during molecular replication

(75) Inventors: Andrew Berlin, San Jose, CA (US); Steven J. Kirch, Pleasanton, CA (US); Gabi Neubauer, Los Gatos, CA (US); Valluri Rao, Saratoga, CA (US); Mineo Yamakawa, Campbell, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Intel Corporation, Santa Clara, CA (US)

(21) Appl. No.: 10/750,315

(22) Filed: Dec. 30, 2003

(51) Int. Cl. C12Q 1/68; C12M 1/34; B05D 3/00

(52) U.S. Cl. 435/6; 427/2.11; 435/287.2

(57) ABSTRACT

The methods and apparatus disclosed herein are useful for detecting nucleotides, nucleosides, and bases and for nucleic acid sequence determination. The methods involve detection of a nucleotide, nucleoside, or base using surface enhanced Raman spectroscopy (SERS). The detection can be part of a nucleic acid sequencing reaction to detect uptake of a deoxynucleotide triphosphate during a nucleic acid polymerization reaction, such as a nucleic acid sequencing reaction. The nucleic acid sequence of a synthesized nascent strand, and the complementary sequence of the template strand, can be determined by tracking the order of incorporation of nucleotides during the polymerization reaction.

### BACKGROUND OF THE INVENTION

1. Field of the Invention

The present methods and apparatus relate to the fields of molecular biology and genomics. More particularly, the disclosed methods and apparatus concern nucleic acid sequencing.

2. Background Information

Genetic information is stored in the form of very long molecules of deoxyribonucleic acid (DNA), organized into chromosomes. The human genome contains approximately three billion bases of DNA sequence. This DNA sequence information determines multiple characteristics of each individual. Many common diseases are based at least in part on variations in DNA sequence.

Determination of the entire sequence of the human genome has provided a foundation for identifying the genetic basis of such diseases. However, a great deal of experimentation remains to be done to identify the genetic variations associated with each disease. This experimentation requires DNA sequencing of portions of chromosomes in individuals or families exhibiting each such disease, in order to identify specific changes in DNA sequence that promote the disease. Ribonucleic acid (RNA), an intermediary molecule in processing genetic information, can also be sequenced to identify the genetic bases of various diseases.

Current sequencing methods require that many copies of a template nucleic acid of interest be produced, cut into overlapping fragments and sequenced, after which the overlapping DNA sequences are assembled into the complete gene. This process is laborious, expensive, inefficient and time-consuming. It also typically requires the use of fluorescent or radioactive labels, which can potentially pose safety and waste disposal problems. Accordingly, a need exists for improved nucleic acid sequencing methods which are less expensive, more efficient, and safer than present methods.

### BRIEF DESCRIPTION OF THE DRAWINGS

FIG. 1 illustrates an exemplary apparatus 10 (not to scale) and method for DNA sequencing in which a nucleic acid 13 is sequenced by monitoring the uptake of nucleotides 17 from solution during nucleic acid synthesis.

FIG. 2 shows the Raman spectra of all four deoxynucleotide monophosphates (dNTPs) at 100 mM concentration, using a 10 second data collection time. Characteristic Raman emission peaks for as shown for each different type of nucleotide. The data were collected without surface-enhancement or labeling of the nucleotides.

FIG. 3 shows SERS detection of 1 nM guanine, obtained from dGMP by acid treatment according to Nucleic Acid Chemistry, Part 1, L. B. Townsend and R. S. Tipson (Eds.), Wiley-Interscience, New York, 1978.

FIG. 4 shows SERS detection of 100 nM cytosine.

FIG. 5 shows SERS detection of 100 nM thymine.

FIG. 6 shows SERS detection of 100 pM adenine.

FIG. 7 shows a comparative SERS spectrum of a 500 rM solution of deoxyadenosine triphosphate covalently labeled with fluorescein (dATP-fluorescein) (upper trace) and unlabeled dATP (lower trace). The dATP-fluorescein was obtained from Roche Applied Science (Indianapolis, Ind.). A strong increase in the SERS signal was detected in the fluorescein labeled dATP.

FIG. 8 shows the SERS detection of a 0.9 nM (nanomolar) solution of adenine. The detection volume was estimated to be about 100 to 150 femtoliters, containing approximately 60 molecules of adenine.

FIG. 9 shows the SERS detection of a rolling circle amplification product, using a single-stranded, circular M13 DNA template.

### DETAILED DESCRIPTION OF THE INVENTION

The disclosed methods and apparatus are useful for the rapid, automated sequencing of nucleic acids. The methods relate to the discovery that a nucleic acid sequencing reaction can be performed by detecting nucleotide uptake of a synthesis reaction using Raman spectroscopy. Advantages over prior art methods include greater speed of obtaining sequence data, decreased cost of sequencing and greater efficiency in operator time required per unit of sequence data, and the ability of reading long nucleic acid sequences in a single sequencing run.

Accordingly, a method for sequencing a nucleic acid is provided, that includes contacting one or more template nucleic acid molecules with nucleotides and a polymerase to form a reaction mixture, and synthesizing one or more complementary strands from the nucleotides, wherein the concentrations of the nucleotides are then measured by Raman spectroscopy. A decrease in the concentration of a nucleotide in the reaction mixture after synthesis of a complementary strand indicates that the nucleotide was incorporated into the complementary strand. The sequence of the template nucleic acid is determined from the nucleotides incorporated into the complementary strand.

In certain aspects, the nucleotides are separated from the template nucleic acid molecules before the nucleotide concentrations are measured, as discussed in more detail herein. Furthermore, a single type of nucleotide can be exposed to the template at one time, or all four types of nucleotides can be exposed to the template simultaneously.

In certain examples, Raman labels are attached to each nucleotide to enhance the Raman signal of the nucleotide, as discussed in further detail herein. Raman labels can be attached to all of the nucleotides, or Raman labels can be attached to only pyrimidine nucleotides, for example. This aspect of the invention relates to data provided in the Examples herein that indicate that under certain conditions more pyrimidines molecules are required to reach a detection limit than purine molecules.

As indicated above, a decrease in the concentration of a nucleotide in the reaction mixture after synthesis of a complementary strand indicates that the nucleotide was incorporated into the complementary strand. A decrease in concentration of a nucleotide can be identified, for example, by identifying a relative decrease in Raman signal generated by the nucleotide after synthesis of the complementary strand compared to a Raman signal obtained from the nucleotide before synthesis of the complementary strand. For example, the Raman signal obtained before synthesis of the complementary strand, can be obtained by generating a Raman signal for the reaction mixture in the absence of template nucleic acid molecules.

In another embodiment, an apparatus that includes a reaction chamber to contain one or more nucleic acid molecules attached to an immobilization surface, a channel in fluid communication with the reaction chamber, and a Raman detection unit operably coupled to the channel, is provided. In certain aspects, the Raman detection unit is capable of detecting at least one nucleotide at the single molecule level. Furthermore, in certain aspects, nucleotides flow through the reaction chamber into the channel, which can include a silver, gold, platinum, copper or aluminum mesh, such as a metal nanoparticle.

In another embodiment, a method for determining a nucleotide sequence of one or more template nucleic acids is provided, that includes contacting the one or more template nucleic acids with a reaction mixture that includes a primer, a polymerase, and an initial concentration of a first nucleotide, and detecting the concentration of the first nucleotide in a post-reaction mixture using Raman spectroscopy, wherein a decrease in the post-reaction concentration of the first nucleotide indicates that the nucleotide was added to the 3′ end of the one or more nascent nucleic acid molecules. Typically, either the template nucleic acid or the primer is immobilized on a solid support, while the template nucleic acid is incubated in the reaction mixture to form a post-reaction mixture and one or more nascent nucleic acid molecule complementary to at least a portion of the template nucleic acid. The above method is optionally repeated with a different nucleotide until the 3′ nucleotide of the one or more nascent nucleic acid molecules is identified, thereby determining a nucleotide sequence of one or more nucleic acid molecules.

In certain aspects, the nucleotide is attached to a Raman label, for example a fluorophore or a nanoparticle, before it is detected by Raman spectroscopy. The Raman spectroscopy can be performed using surface enhanced Raman spectroscopy (SERS), for example.

A template molecule is isolated, in certain aspects, from a biological sample, before it is detected by the methods disclosed herein. The biological sample is, for example, urine, blood, plasma, serum, saliva, semen, stool, sputum, cerebral spinal fluid, tears, mucus, and the like.

In certain aspects, the biological sample is from a mammalian subject, for example a human subject. The biological sample can be virtually any biological sample, particularly a sample that contains RNA or DNA from a subject. The biological sample can be a tissue sample which contains, for example, 1 to 10,000,000; 1000 to 10,000,000; or 1,000,000 to 10,000,000 somatic cells. The sample need not contain intact cells, as long as it contains sufficient RNA or DNA for the methods of the present invention, which in some aspects require only 1 molecule of RNA or DNA. According to aspects of the present invention wherein the biological sample is from a mammalian subject, the biological or tissue sample can be from any tissue. For example, the tissue can be obtained by surgery, biopsy, swab, stool, or other collection method.

In other aspects, the biological sample contains a pathogen, for example a virus or a bacterial pathogen. In certain aspects, the template nucleic acid is purified from the biological sample before it is contacted with a probe, however. The isolated template nucleic acid can be contacted with a reaction mixture without being amplified.

In another embodiment, a method for determining a nucleotide occurrence at a target position of a template nucleic acid molecule is provided, that includes contacting the template nucleic acid with a reaction mixture that includes a primer, a polymerase, and an initial concentration of a first nucleotide to form a post-reaction mixture, wherein the 3′ nucleotide of the primer binds to the template nucleic acid adjacent to the target nucleotide position, and determining the concentration of the first nucleotide in the post-reaction mixture using Raman spectroscopy, wherein a decrease in the post-reaction concentration of the first nucleotide identifies an extension reaction product, and indicates that the nucleotide is complementary to the nucleotide at the target position, thereby identifying the nucleotide occurrence at the target position. Typically, either the target nucleic acid molecule or the primer are immobilized on a substrate. The method is optionally repeated with a different nucleotide until the nucleotide occurrence is identified.

The target position, for example, can be a site of a polymorphism, such as a single nucleotide polymorphism (SNP). Polymorphisms are allelic variants that occur in a population. A polymorphism can be a single nucleotide difference present at a locus, or can be an insertion or deletion of one or a few nucleotides. As such, a single nucleotide polymorphism (SNP) is characterized by the presence in a population of one or two, three or four nucleotide occurrences (i.e., adenosine, cytosine, guanosine or thymidine) at a particular locus in a genome such as the human genome. As indicated herein, methods of the invention in certain aspects, provide for the detection of a nucleotide occurrence at a SNP location or a detection of both genomic nucleotide occurrences at a SNP location for a diploid organism such as a mammal.

In another embodiment, a method for detecting a nucleotide, nucleoside, or base is provided, wherein the nucleotide, nucleoside, or base are deposited on a substrate that includes metallic nanoparticles, a metal-coated nanostructure, or a substrate that includes aluminum, before irradiated the deposited nucleotide, nucleoside or base with a laser beam, and detecting the resulting Raman spectra. The detection method is useful, for example, in methods of sequencing nucleic acids disclosed herein.

The nucleotide, nucleoside, or base in certain examples is deposited on one or more silver nanoparticles between about 5 and 200 nm in diameter. For example, the nucleotide, nucleoside, or base is deposited on silver nanoparticles. In these aspects, for example, the nucleotide, nucleoside, or base can be contacted with an alkali-metal halide salt and the silver nanoparticles. The alkali-metal halide salt is, for example, lithium chloride. In these aspects, for example, lithium chloride can be used at a concentration of about 50 to about 150 micromolar, about 80 to about 100 micromolar, or about 90 micromolar.

The nucleotide, nucleoside, or base in certain aspects, includes adenine, and in certain examples, a single molecule of adenine is detected. The base can be associated with a Raman label, in certain examples.

In another embodiment, a method of sequencing nucleic acids is provided, that includes obtaining one or more template nucleic acid molecules and providing nucleotides and a polymerase to the template to allow synthesis of one or more complementary strands using the nucleotides, and measuring the concentrations of the nucleotides using Raman spectroscopy. The sequence of the template nucleic acid is determined from the nucleotides incorporated into the complementary strand.

In another embodiment, an apparatus that includes a reaction chamber containing a single template nucleic acid molecule or primer attached to an immobilization surface; a channel in fluid communication with the reaction chamber; and a Raman detection unit operably coupled to the channel, is provided.

Sequence information using the methods of the present invention can be obtained during the course of a single sequencing run, using a single nucleic acid molecule. Alternatively, multiple copies of a nucleic acid molecule can be sequenced in parallel or sequentially to confirm the nucleic acid sequence or to obtain complete sequence data. In other alternatives, both the nucleic acid molecule and its complementary strand can be sequenced to confirm the accuracy of the sequence information. The nucleic acid to be sequenced can be DNA, although other nucleic acids including RNA or synthetic nucleotide analogs can also be sequenced.

A nucleic acid to be sequenced can be attached, either covalently or non-covalently to a surface. Alternatively, a nucleic acid to be sequenced can be restricted in location by non-attachment methods, such as optical trapping (see, e.g., Goodwin et al., 1996, Acc. Chem. Res. 29:607-619; U.S. Pat. Nos. 4,962,037; 5,405,747; 5,776,674; 6,136,543; 6,225,068). Attachment or other localization of the nucleic acid allows the nucleotides to be detected by Raman spectroscopy without background signals from the nucleic acid. For example, a continuous or discontinuous flow of nucleotides can be provided for nucleic acid synthesis. The concentrations of nucleotides can be determined upstream and downstream of the synthetic reaction. The difference in nucleotide concentration represents the nucleotides that have been incorporated into a newly synthesized complementary nucleic acid strand. Alternatively, a nucleic acid template, primer and polymerase can be restricted to a subcompartment of a reaction chamber. The Raman detector can be arranged to detect nucleotide concentrations in a different portion of the reaction chamber, without background signals from the nucleic acid, polymerase and primer. Nucleotides can be allowed to equilibrate between the different parts of the reaction chamber by passive diffusion or active mixing processes.

The following detailed description contains numerous specific details in order to provide a more thorough understanding of the claimed methods and apparatus. However, it will be apparent to those skilled in the art that the apparatus and/or methods can be practiced without these specific details. In other instances, those devices, methods, procedures, and individual components that are well known in the art have not been described in detail herein.

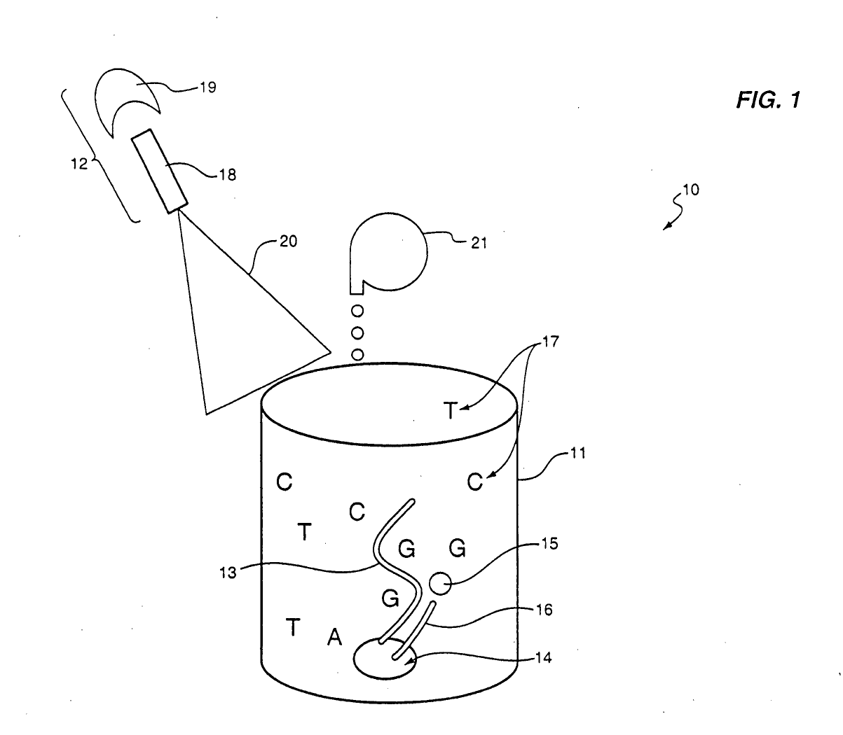

FIG. 1 illustrates a non-limiting example of an apparatus 10 for nucleic acid sequencing, that includes a reaction chamber 11 and a Raman detection unit 12. The reaction chamber 11 contains a nucleic acid (template) molecule 13 attached to an immobilization surface 14 along with a polymerase 15, such as a DNA polymerase. A primer molecule 16 that is complementary in sequence to the template molecule 13 is allowed to hybridize to the template molecule 13. Nucleotides 17 are present in solution in the reaction chamber 11. For synthesis of a nascent DNA strand 16, the nucleotides 17 can include deoxyadenosine-5′-triphosphate (dATP), deoxyguanosine-5′-triphosphate (dGTP), deoxycytosine-5′-triphosphate (dCTP) and/or deoxythymidine-5′-triphosphate (dTTP). Each of the four nucleotides 17 can be present simultaneously in solution. Alternatively, different types of nucleotides 17 can be sequentially added to the reaction chamber 11. Furthermore, other nucleotides such as uridine-5′-triphosphate (UTP) can be utilized, especially where the nascent strand is an RNA molecule. Non-natural nucleotides, such as those used in traditional nucleic acid sequencing, can also be used. These include all fluorescent dyes-labeled nucleotides (e.g., Cy 3, Cy3.5, Cy5, Cy5.5, TAMRA, R6G (available, for example, from Applied Biosystems, Foster City, Calif.; or NEN Life Science Products, Boston, Mass.) that have been used by the standard sequencing or labeling reactions. These dyes can be detected by SERS.

To initiate a sequencing reaction, a polymerase 15 adds one nucleotide molecule 17 at a time to the 3′ end of the primer 16, elongating the primer molecule 16. As the primer molecule 16 is extended, it is referred to as a nascent strand 16. For each round of elongation, a single nucleotide 17 is incorporated into the nascent strand 16. Because incorporation of nucleotides 17 is determined by Watson-Crick base pair interactions with the template strand 13, the sequence of the growing nascent strand 16 will be complementary to the sequence of the template strand 13. In Watson-Crick base pairing, an adenosine (A) residue on one strand is paired with a thymidine (T) residue on the other strand. Similarly, a guanosine (G) residue on one strand is paired with a cytosine (C) residue on the other strand. Thus, the sequence of the template strand 13 can be determined from the sequence of the nascent strand 16.

FIG. 1 illustrates a method and apparatus 10 in which a single nucleic acid molecule 13 is contained in a reaction chamber 11. Alternatively, two or more template nucleic acid molecules 13 of identical sequence can be present in a single reaction chamber 11. Where more than one template nucleic acid 13 is present in the reaction chamber 11, the Raman emission signals will reflect an average of the nucleotides 17 incorporated into all nascent strands 16 in the reaction chamber 11. The skilled artisan will be able to correct the signal obtained at any given time for synthetic reactions that either lag behind or precede the majority of reactions occurring in the reaction chamber 11, using known data analysis techniques.

The non-limiting example illustrated in FIG. 1 shows the nucleotides 17 to be detected by Raman spectroscopy in the same reaction chamber 11 as the template strand 13, primer 16 and polymerase 15. To reduce interfering Raman signals, the reaction chamber 11 and detection unit 12 can be arranged so that only nucleotides 17 are excited and detected. For example, the reaction chamber 11 can be divided into two parts, with the template 13, primer 16 and polymerase 15 confined to one part of the chamber 11 by immobilization on a surface 14, by use of a low molecular weight cutoff filter, by optical trapping or by other methods known in the art (see, e.g., Goodwin et al., 1996, Acc. Chem. Res. 29:607-619; U.S. Pat. Nos. 4,962,037; 5,405,747; 5,776,674; 6,136,543; 6,225,068). The detection unit 12 can be arranged so that only nucleotides 17 in the second part of the chamber 11 are excited to emit Raman signals. Alternatively, the reaction chamber 11 can be attached to a flow-through system, such as a microfluidic channel, microcapillary or nanochannel. Nucleotides 17 can enter the reaction chamber 11 and be incorporated into a nascent strand 16. Residual unincorporated nucleotides 17 can pass out of the reaction chamber 11 into a second channel, where they are detected by Raman spectroscopy. The template 13, primer 16 and polymerase 15 can be confined to the reaction chamber 11 by attachment, use of a filter, optical trapping or other known methods. Because the nucleotides 17 are detected in a separate compartment from the template 13, primer 16 and polymerase 15, interfering Raman signals are minimized. The nucleotides 17 incorporated into the nascent strand 16 can be identified by the difference in concentration of nucleotides 17 entering the reaction chamber 11 and leaving the reaction chamber 11. In such alternatives, duplicate detection units 12 can be positioned before and after the reaction chamber 11. Alternatively, where the concentrations of nucleotides 17 entering the reaction chamber 11 are known, a single detection unit 12 can be positioned downstream of the reaction chamber 11 to measure nucleotide 17 concentrations exiting the reaction chamber 11.

The skilled artisan will realize that depending on the type of polymerase 15 used, the nascent strand 16 can contain some percentage of mismatched bases, where the newly incorporated base is not correctly hydrogen bonded with the corresponding base in the template strand 13. An accuracy of at least 90%, at least 95%, at least 98%, at least 99%, at least 99.5%, at least 99.8%, at least 99.9% or higher can be observed. Certain polymerases 15 are known to have an error correction activity (also referred to as a 3′ exonuclease or proof-reading activity) that acts to remove a newly incorporated nucleotide 17 that is incorrectly base-paired to the template strand 13. Polymerases 15 with or without a proof-reading activity can be employed in the disclosed methods. A polymerase 15 with the lowest possible error rate can be used for specific applications. Polymerase 15 error rates are known in the art.

The detection unit 12 includes an excitation source 18, such as a laser, and a Raman spectroscopy detector 19. The excitation source 18 illuminates the reaction chamber 11 or channel with an excitation beam 20. The excitation beam 20 interacts with the nucleotides 17, resulting in the excitation of electrons to a higher energy state. As the electrons return to a lower energy state, they emit a Raman emission signal that is detected by the Raman detector 19. Because the Raman emission signal from each of the four types of nucleotide 17 can be distinguished, the detection unit 12 is capable of measuring the amount of each type of nucleotide 17 in the reaction chamber 11 and/or channel.

The incorporation of nucleotides 17 into the growing nascent strand 16 results in a depletion of nucleotides 17 from the reaction chamber 11. In order for the synthetic reaction to continue, a source of fresh nucleotides 17 can be required. This source is illustrated in FIG. 1 as a molecule dispenser 21. A molecule dispenser 21 can or can not be part of the sequencing apparatus 10.

The molecule dispenser 21 can be designed to release each of the four nucleotides 17 in equal amounts, calibrated to the rate of synthesis of the nascent strand 16. However, nucleic acids 13 do not necessarily exhibit a uniform distribution of A, T, G and C residues. In particular, certain regions of DNA molecules 13 can be either AT rich or GC rich, depending on the species from which the DNA 13 is obtained and the specific region of the DNA molecule 13 being sequenced. The release of nucleotides 17 from the molecule dispenser 21 can be controlled so that relatively constant concentrations of each type of nucleotide 17 are maintained in the reaction chamber 11.

Data can be collected from a detector 19, such as a spectrometer or a monochromator array and provided to an information processing and control system. The information processing and control system can maintain a database associating specific Raman signatures with specific nucleotides 17. The information processing and control system can record the signatures detected by the detector 19 and can correlate those signatures with the signatures of known nucleotides 17. The information processing and control system can also maintain a record of nucleotide 17 uptake that indicates the sequence of the template molecule 13. The information processing and control system can also perform standard procedures known in the art, such as subtraction of background signals.

Where the nascent strand 16 includes DNA, the template strand 13 can be either RNA or DNA. With an RNA template strand 13, the polymerase 15 can be a reverse transcriptase, examples of which are known in the art. Where the template strand 13 is a molecule of DNA, the polymerase 15 can be a DNA polymerase.

Alternatively, the nascent strand 16 can be a molecule of RNA. This requires that the polymerase 15 be an RNA polymerase, for which no primer 16 is required. However, the template strand 13 should contain a promoter sequence that is effective to bind RNA polymerase 15 and initiate transcription of an RNA nascent strand 16. The exact composition of the promoter sequence depends on the type of RNA polymerase 15 used. Optimization of promoter sequences to allow for efficient initiation of transcription is within the routine skill in the art. The methods are not limited as to the type of template molecule 13 used, the type of nascent strand 16 synthesized, or the type of polymerase 15 utilized. Virtually any template 13 and any polymerase 15 that can support synthesis of a nucleic acid molecule 16 complementary in sequence to the template strand 13 can be used.

The nucleotides 17 can be chemically modified with a Raman label. The label can have a unique and highly visible optical signature that can be distinguished for each of the common nucleotides 17. The label can also serve to increase the strength of the Raman emission signal or to otherwise enhance the sensitivity or specificity of the Raman detector 19 for nucleotides 17. Non-limiting examples of tag molecules that could be used for Raman spectroscopy are disclosed below. The use of labels in Raman spectroscopy is known in the art (e.g., U.S. Pat. Nos. 5,306,403 and 6,174,677). The skilled artisan will realize that Raman labels can generate distinguishable Raman spectra when bound to different nucleotides 17, or different labels can be designed to bind only one type of nucleotide 17.

The template molecule 13 can be attached to a surface 14 such as functionalized glass, silicon, PDMS (polydimethlyl siloxane), silver or other metal coated surfaces, quartz, plastic, PTFE (polytetrafluoroethylene), PVP (polyvinyl pyrrolidone), polystyrene, polypropylene. polyacrylamide, latex, nylon, nitrocellulose, a glass bead, a magnetic bead, or any other material known in the art that is capable of having functional groups such as amino, carboxyl, thiol, hydroxyl or Diels-Alder reactants incorporated on its surface.

Functional groups can be covalently attached to cross-linking agents so that binding interactions between template strand 13 and polymerase 15 can occur without steric hindrance. Typical cross-linking groups include ethylene glycol oligomers and diamines. Attachment can be by either covalent or non-covalent binding. Various methods of attaching nucleic acid molecules 13 to surfaces 14 are known in the art and can be employed.

As used herein, “a” or “an” can mean one or more than one of an item.

“Nucleic acid” means either DNA, RNA, single-stranded, double-stranded or triple stranded and any chemical modifications thereof. Virtually any modification of the nucleic acid is contemplated. A “nucleic acid” can be of almost any length, from 10, 20, 30, 40, 50, 60, 75. 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100, 000, 150, 000, 200, 000, 500,000, 1,000,000, 1,500,000, 2,000,000, 5,000,000 or even more bases in length, up to a full-length chromosomal DNA molecule.

A nucleoside is a molecule that includes a purine or pyrimidine base covalently attached to a pentose sugar such as deoxyribose, ribose or derivatives or analogs of pentose sugars. A “nucleotide” refers to a nucleoside further including at least one phosphate group covalently attached to the pentose sugar. It is contemplated that various substitutions or modifications can be made in the structure of the nucleotides, so long as they are still capable of being incorporated into a nascent strand by the polymerase. For example, the ribose or deoxyribose moiety can be substituted with another pentose sugar or a pentose sugar analog. The phosphate groups can be substituted by various groups, such as phosphonates, sulphates or sulfonates. The naturally occurring purine or pyrimidine bases can be substituted by other purines or pyrimidines or analogs thereof, so long as the sequence of nucleotides incorporated into the nascent strand reflects the sequence of the template strand.

Template molecules can be prepared by any technique known to one of ordinary skill in the art. The template molecules can be naturally occurring DNA or RNA molecules, for example, chromosomal DNA or messenger RNA (mRNA). Virtually any naturally occurring nucleic acid can be prepared and sequenced by the disclosed methods including, without limit, chromosomal, mitochondrial or chloroplast DNA or ribosomal, transfer, heterogeneous nuclear or messenger RNA. Nucleic acids to be sequenced can be obtained from either prokaryotic or eukaryotic sources by standard methods known in the art.

Methods for preparing and isolating various forms of cellular nucleic acids are known (see, e.g., Guide to Molecular Cloning Techniques, eds. Berger and Ibmmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989). Generally, cells, tissues or other source material containing nucleic acids to be sequenced are first homogenized, for example by freezing in liquid nitrogen followed by grinding in a mortar and pestle. Certain tissues can be homogenized using a Waring blender, Virtis homogenizer, Dounce homogenizer or other homogenizer. Crude homogenates can be extracted with detergents, such as sodium dodecyl sulfate (SDS), Triton X-100, CHAPS (3-[(3-cholamidopropyl)-dimethylammonio]-1-propane sulfonate), octylglucoside or other detergents known in the art. Alternatively or in addition, extraction can use chaotrophic agents such as guanidinium isothiocyanate, or organic solvents such as phenol. Protease treatment, for example with proteinase K, can be used to degrade cell proteins. Particulate contaminants can be removed by centrifugation or ultracentrifugation (for example, 10 to 30 min at about 5,000 to 10,000×g, or 30 to 60 min at about 50,000 to 100,000×g). Dialysis against aqueous buffer of low ionic strength can be of use to remove salts or other soluble contaminants. Nucleic acids can be precipitated by addition of ethanol at −20° C., or by addition of sodium acetate (pH 6.5, about 0.3 M) and 0.8 volumes of 2-propanol. Precipitated nucleic acids can be collected by centrifugation or, for chromosomal DNA, by spooling the precipitated DNA on a glass pipette or other probe.

The skilled artisan will realize that the procedures listed above are exemplary only and that many variations can be used, depending on the particular type of nucleic acid to be sequenced. For example, mitochondrial DNA is often prepared by cesium chloride density gradient centrifugation, using step gradients, while mRNA is often prepared using preparative columns from commercial sources, such as Promega (Madison, Wis.) or Clontech (Palo Alto, Calif.). Such variations are known in the art.

The skilled artisan will realize that depending on the type of template nucleic acid to be prepared, various nuclease inhibitors can be used. For example, RNase contamination in bulk solutions can be eliminated by treatment with diethyl pyrocarbonate (DEPC), while commercially available nuclease inhibitors can be obtained from standard sources such as Promega (Madison, Wis.) or BRL (Gaithersburg, Md.). Purified nucleic acid can be dissolved in aqueous buffer, such as TE (Tris-EDTA) (ethylene diamine tetraacetic acid) and stored at −20° C. or in liquid nitrogen prior to use.

In cases where single stranded DNA (ssDNA) is to be sequenced, ssDNA can be prepared from double stranded DNA (dsDNA) by standard methods. Most simply, dsDNA can be heated above its annealing temperature, at which point it spontaneously separates into ssDNA. Representative conditions might involve heating at 92 to 95° C. for 5 min or longer. Formulas for determining conditions to separate dsDNA, based for example on GC content and the length of the molecule, are known in the art. Alternatively, single-stranded DNA can be prepared from double-stranded DNA by standard amplification techniques known in the art, using a primer that only binds to one strand of double-stranded DNA. Other methods of preparing single-stranded DNA are known in the art, for example by inserting the double-stranded nucleic acid to be sequenced into the replicative form of a phage like M13, and allowing the phage to produce single-stranded copies of the template.

Virtually any type of nucleic acid that can serve as a template for an RNA or DNA polymerase can potentially be sequenced. For example, nucleic acids prepared by various amplification techniques, such as polymerase chain reaction (PCRTM) amplification, can be sequenced (see U.S. Pat. Nos. 4,683,195, 4,683,202 and 4,800,159). Nucleic acids to be sequenced can alternatively be cloned in standard vectors, such as plasmids, cosmids, BACs (bacterial artificial chromosomes) or YACs (yeast artificial chromosomes) (see, e.g., Berger and Kimmel, 1987; Sambrook et al., 1989). Nucleic acid inserts can be isolated from vector DNA, for example, by excision with appropriate restriction endonucleases, followed by agarose gel electrophoresis and ethidium bromide staining. Selected size-fractionated nucleic acids can be removed from gels, for example by the use of low melting point agarose or by electroelution from gel slices. Methods for insert isolation are known to the person of ordinary skill in the art.

In certain aspects, nucleic acids to be sequenced can be a single molecule of ssDNA or ssRNA. For aspects in which a small number (e.g. 1000 molecules or less) of nucleic acid templates are included, procedures for minimizing binding of nucleic acids to surfaces of reaction vessels and substrates can be included, such as by adding negative charges to surfaces (see, e.g., Braslavsky et al., Proc. Natl. Acad. Sci., 100:3960-3964, 2003).

A variety of methods for selection and manipulation of single ssDNA or ssRNA molecules can be used, for example, hydrodynamic focusing, micro-manipulator coupling, optical trapping, or combination of these and similar methods (see, e.g., Goodwin et al., 1996, Acc. Chem. Res. 29:607-619; U.S. Pat. Nos. 4,962,037; 5,405,747; 5,776,674; 6,136,543; 6,225,068).

In particular embodiments of the invention, the methods and apparatus are suitable for obtaining the sequences of very long nucleic acid molecules of greater than 1,000, greater than 2,000, greater than 5,000, greater than 10,000 greater than 20,000, greater than 50,000, greater than 100,000 or even more bases in length. However, in certain embodiments, the methods and apparatus provide the sequence of a shorter nucleic acid molecule that is 500, 400, 300, 200, 150, 100, 50, 25, 20, 15, 10, 9, 8, 7, 6, 5, 4, 3, 2, or 1 nucleotide in length. This shorter nucleic acid molecule can be isolated directly from a sample or can result from processing of longer nucleic acid molecules.

Microfluidics or nanofluidics can be used to sort and isolate template nucleic acids. Hydrodynamics can be used to manipulate nucleic acids into a microchannel, microcapillary, or a micropore. Hydrodynamic forces can be used to move nucleic acid molecules across a comb structure to separate single nucleic acid molecules. Once the nucleic acid molecules have been separated, hydrodynamic focusing can be used to position the molecules. A thermal or electric potential, pressure or vacuum can also be used to provide a motive force for manipulation of nucleic acids. Manipulation of template nucleic acids for sequencing can involve the use of a channel block design incorporating microfabricated channels and an integrated gel material, as disclosed in U.S. Pat. Nos. 5,867,266 and 6,214,246.

Alternatively, a sample containing a nucleic acid template can be diluted prior to coupling to an immobilization surface. The immobilization surface can be in the form of magnetic or non-magnetic beads or other discrete structural units. At an appropriate dilution, each bead will have a statistical probability of binding zero or one nucleic acid molecules. Beads with one attached nucleic acid molecule can be identified using, for example, fluorescent dyes and flow cytometer sorting or magnetic sorting. Depending on the relative sizes and uniformity of the beads and the nucleic acids, it can be possible to use a magnetic filter and mass separation to separate beads containing a single bound nucleic acid molecule. In other alternatives, multiple nucleic acids attached to a single bead or other immobilization surface can be sequenced.

In further alternatives, a coated fiber tip can be used to generate single molecule nucleic acid templates for sequencing (e.g., U.S. Pat. No. 6,225,068). The immobilization surfaces can be prepared to contain a single molecule of avidin or other cross-linking agent. Such a surface could attach a single biotinylated primer, which in turn can hybridize with a single template nucleic acid to be sequenced. This is not limited to the avidin-biotin binding system, but can be adapted to any coupling system known in the art.

In other alternatives, an optical trap can be used for manipulation of single molecule nucleic acid templates for sequencing. (E.g., U.S. Pat. No. 5:776.674). Exemplary optical trapping systems are commercially available from Cell Robotics, Inc. (Albuquerque, N.Mex.), S+L GmbH (Heidelberg, Germany) and P.A.L.M. Gmbh (Wolfratshausen, Germany).

The nucleic acid molecules to be sequenced can be attached to a solid surface (or immobilized). Immobilization of nucleic acid molecules can be achieved by a variety of methods involving either non-covalent or covalent attachment between the nucleic acid molecule and the surface. For example, immobilization can be achieved by coating a surface with streptavidin or avidin and the subsequent attachment of a biotinylated polynucleotide (Holmstrom et al., Anal. Biochem. 209:278-283, 1993). Immobilization can also occur by coating a silicon, glass or other surface with poly-L-Lys (lysine), followed by covalent attachment of either amino- or sulfhydryl-modified nucleic acids using bifunctional cross-linking reagents (Running et al., BioTechniques 8:276-277, 1990; Newton et al., Nucleic Acids Res. 21:1155-62, 1993). Amine residues can be introduced onto a surface through the use of aminosilane for cross-linking.

Immobilization can take place by direct covalent attachment of 5′-phosphorylated nucleic acids to chemically modified surfaces (Rasmussen et al., Anal. Biochem. 198:138-142, 1991). The covalent bond between the nucleic acid and the surface is formed by condensation with a water-soluble carbodiimide. This method facilitates a predominantly 5′-attachment of the nucleic acids via their 5′-phosphates.

DNA is commonly bound to glass by first silanizing the glass surface, then activating with carbodiimide or glutaraldehyde. Alternative procedures can use reagents such as 3-glycidoxypropyltrimethoxysilane (GOP) or aminopropyltrimethoxysilane (APTS) with DNA linked via amino linkers incorporated either at the 3′ or 5′ end of the molecule. DNA can be bound directly to membrane surfaces using ultraviolet radiation. Other non-limiting examples of immobilization techniques for nucleic acids are disclosed in U.S. Pat. Nos. 5,610,287, 5,776,674 and 6,225,068.

The type of surface to be used for immobilization of the nucleic acid is not limiting. The immobilization surface can be magnetic beads, non-magnetic beads, a planar surface, a pointed surface, or any other conformation of solid surface that includes almost any material, so long as the material is sufficiently durable and inert to allow the nucleic acid sequencing reaction to occur. Non-limiting examples of surfaces that can be used include glass, silica, silicate, PDMS, silver or other metal coated surfaces, nitrocellulose, nylon, activated quartz, activated glass, polyvinylidene difluoride (PVDF), polystyrene, polyacrylamide, other polymers such as poly(vinyl chloride), poly(methyl methacrylate) or poly(dimethyl siloxane), and photopolymers which contain photoreactive species such as nitrenes, carbenes and ketyl radicals capable of forming covalent links with nucleic acid molecules (see, e.g., U.S. Pat. Nos. 5,405,766 and 5,986,076).

Bifunctional cross-linking reagents can be of use for attaching a nucleic acid molecule to a surface. The bifunctional cross-linking reagents can be divided according to the specificity of their functional groups, e.g., amino, guanidino, indole, or carboxyl specific groups. Of these, reagents directed to free amino groups are popular because of their commercial availability, ease of synthesis and the mild reaction conditions under which they can be applied. Exemplary methods for cross-linking molecules are disclosed in U.S. Pat. Nos. 5,603,872 and 5,401,511. Cross-linking reagents include glutaraldehyde (GAD), bifunctional oxirane (OXR), ethylene glycol diglycidyl ether (EGDE), and carbodiimides, such as 1-ethyl-3-(3-dimethylaminopropyl) carbodiimide (EDC).

In certain methods, the sequencing reaction can involve binding of a polymerase, such as a DNA polymerase, to a primer molecule and the catalyzed addition of nucleotides to the 3′ end of the primer. Non-limiting examples of polymerases of potential use include DNA polymerases, RNA polymerases, reverse transcriptases, and RNA-dependent RNA polymerases. The differences between these polymerases in terms of their “proofreading” activity and requirement or lack of requirement for primers and promoter sequences are discussed herein and are known in the art. Where RNA polymerases are used, the template molecule to be sequenced can be double-stranded DNA. Errors due to incorporation of mismatched nucleotides can be corrected, for example, by sequencing both strands of the original template, or by sequencing multiple copies of the same strand.

Non-limiting examples of polymerases that can be used include Thermatoga maritima DNA polymerase, AmplitaqFS™ DNA polymerase, Taquenase™ DNA polymerase, ThermoSequenase™, Taq DNA polymerase, Qbeta T replicase, T4 DNA polymerase, Thermus thermophilus DNA polymerase, RNA-dependent RNA polymerase and SP6 RNA polymerase.

A number of polymerases are commercially available, including Pwo DNA Polymerase from Boehringer Mannheim Biochemicals (Indianapolis, Tenn.); Bst Polymerase from Bio-Rad Laboratories (Hercules, Calif.); IsoTherm™ DNA Polymerase from Epicentre Technologies (Madison, Wis.); Moloney Murine Leukemia Virus Reverse Transcriptase, Pfu DNA Polymerase, Avian Myeloblastosis Virus Reverse Transcriptase, Thermus flavus (Tfl) DNA Polymerase and Thermococcus litoralis (Tli) DNA Polymerase from Promega (Madison, Wis.); RAV2 Reverse Transcriptase, HIV-1 Reverse Transcriptase, T7 RNA Polymerase, T3 RNA Polymerase, SP6 RNA Polymerase, RNA Polymerase E. coli, Thermus aquaticus DNA Polymerase, T7 DNA Polymerase +/−3′→3 5′ exonuclease, Klenow Fragment of DNA Polymerase I, Thermus ‘ubiquitous’ DNA Polymerase, and DNA polymerase I from Amersham Pharmacia Biotech (Piscataway, N.J.). However, any polymerase that is known in the art for the template dependent polymerization of nucleotides can be used (see, e.g., Goodman and Tippin, Nat. Rev. Mol. Cell Biol. 1(2):101-9, 2000; U.S. Pat. No. 6,090,389).

The skilled artisan will realize that the rate of polymerase activity can be manipulated to coincide with the optimal rate of analysis of nucleotides by the detection unit. Various methods are known for adjusting the rate of polymerase activity, including adjusting the temperature, pressure, pH, salt concentration, divalent cation concentration, or the concentration of nucleotides in the reaction chamber. Methods of optimization of polymerase activity are known to the person of ordinary skill in the art.

Primers can be obtained by any method known in the art. Generally, primers are between ten and twenty bases in length, although longer primers can be employed. Primers can be designed to be exactly complementary in sequence to a known portion of a template nucleic acid molecule, which can be close to the attachment site of the template to the immobilization surface. Methods for synthesis of primers of any sequence, for example using an automated nucleic acid synthesizer employing phosphoramidite chemistry are known and such instruments can be obtained from standard sources, such as Applied Biosystems (Foster City, Calif.).

Other methods involve sequencing a nucleic acid in the absence of a known primer binding site. In such cases, it can be possible to use random primers, such as random hexamers or random oligomers of 7, 8, 9, 10, 11, 12, 13, 14, 15 bases or greater length, to initiate polymerization of a nascent strand. Non-hybridized primers can be removed before initiating the synthetic reaction.

Non-hybridized primer removal can be accomplished, for example, by using an immobilization surface coated with a binding agent, such as streptavidin. A complementary binding agent, such as biotin, can be attached to the 5′ end of the template molecules, and the template molecules can be immobilized on the immobilization surface. After allowing hybridization between primer and template to occur, those primer molecules that are not also bound to the immobilization surface can be removed. Only those primers that are hybridized to the template strand will serve as primers for template dependent DNA synthesis. In other alternative embodiments, multiple primer molecules can be attached to the immobilization surface. A template molecule can be added and allowed to hydrogen bond to a complementary primer. A template dependent polymerase can then act to initiate nascent strand synthesis.

Other types of cross-linking can be used to selectively retain only one primer per template strand, such as photoactivatable cross-linkers. As discussed above, a number of cross-linking agents are known in the art and can be used. Cross-linking agents can also be attached to the immobilization surface through linker arms, to avoid the possibility of steric hindrance with the immobilization surface interfering with hydrogen bonding between the primer and template.

Certain methods can involve incorporating a label into the nucleotides, to facilitate their measurement by the detection unit. A Raman label can be any organic or inorganic molecule, atom, complex or structure capable of producing a detectable Raman signal, including but not limited to synthetic molecules, dyes, naturally occurring pigments such as phycoerythrin, organic nanostructures such as C₆₀, buckyballs and carbon nanotubes, metal nanostructures such as gold or silver nanoparticles or nanoprisms and nano-scale semiconductors such as quantum dots. Numerous examples of Raman labels are disclosed below. The skilled artisan will realize that such examples are not limiting, and that a Raman label can encompasses any organic or inorganic atom, molecule, compound or structure known in the art that can be detected by Raman spectroscopy.

Non-limiting examples of labels that can be used for Raman spectroscopy include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanines, azomethines, cyanines, xanthines, succinylfluoresceins and aminoacridine. Polycyclic aromatic compounds in general can function as Raman labels, as is known in the art. These and other Raman labels can be obtained from commercial sources (e.g., Molecular Probes, Eugene, Oreg.).

Other labels that can be of use include cyanide, thiol, chlorine, bromine, methyl, phosphorus and sulfur. Carbon nanotubes can also be of use as Raman labels. The use of labels in Raman spectroscopy is known (e.g., U.S. Pat. Nos. 5,306,403 and 6,174,677). The skilled artisan will realize that Raman labels should generate distinguishable Raman spectra when bound to different types of nucleotide.

Labels can be attached directly to the nucleotides or can be attached via various linker compounds. Alternatively, nucleotide precursors that are covalently attached to Raman labels are available from standard commercial sources (e.g., Roche Molecular Biochemicals, Indianapolis, Ind.; Promega Corp., Madison, Wis.; Ambion, Inc., Austin, Tex.; Amersham Pharmacia Biotech, Piscataway, N.J.). Raman labels that contain reactive groups designed to covalently react with other molecules, such as nucleotides, are commercially available (e.g., Molecular Probes, Eugene, Oreg.). Methods for preparing labeled nucleotides and incorporating them into nucleic acids are known (e.g., U.S. Pat. Nos. 4,962,037; 5,405,747; 6,136,543; 6,210,896). In certain aspects of the present invention Raman labels are attached to the pyrimidine nucleotides.

An apparatus according to the present invention includes a channel. In certain aspects, the concentrations of nucleotides is measured by Raman spectroscopy as they flow through the channel. The channel in certain aspects includes a silver, gold, platinum, copper or aluminum mesh. The channel is, for example, a microfluidic channel, a microchannel, a microcapillary or a nanochannel. Furthermore, the reaction chamber and the channel in certain examples are incorporated into a single chip.

The apparatus further includes, in certain examples, metal nanoparticles in the channel. The nanoparticles flow through the channel in certain aspects of the invention. The channel diameter, in certain aspects, is about 5, 10, 20, 25, 30, 40, 50, 75, 100, 125, 150, 175, 200, 225, 250, 275 and 300 micrometers. For example, the channel diameter is between about 100 and about 200 micrometers in diameter. In other aspects, the channel is round.

An apparatus of the present invention, typically includes a reaction chamber. A reaction chamber can be designed to hold an immobilization surface, nucleic acid template, primer, polymerase and/or nucleotides in an aqueous environment. The reaction chamber can be designed to be temperature controlled, for example by incorporation of Pelletier elements or other methods known in the art. Methods of controlling temperature for low volume liquids used in nucleic acid polymerization are known in the art (see, e.g., U.S. Pat. Nos. 5,038,853, 5,919,622, 6,054,263 and 6,180,372).

The reaction chamber and any associated fluid channels, for example, to provide connections to a molecule dispenser, to a detection unit, to a waste port, to a loading port, or to a source of nucleotides can be manufactured in a batch fabrication process, as known in the fields of computer chip manufacture or microcapillary chip manufacture. The reaction chamber and other components of the apparatus can be manufactured as a single integrated chip. Such a chip can be manufactured by methods known in the art: such as by photolithography and etching, laser ablation, injection molding, casting, molecular beam epitaxy, dip-pen nanolithography, chemical vapor deposition (CVD) fabrication, electron beam or focused ion beam technology or imprinting techniques. Non-limiting examples include conventional molding with a flowable, optically clear material such as plastic or glass; photolithography and dry etching of silicon dioxide; electron beam lithography using polymethylmethacrylate resist to pattern an aluminum mask on a silicon dioxide substrate, followed by reactive ion etching. Microfluidic channels can be made by molding polydimethylsiloxane (PDMS) according to Anderson et al. (“Fabrication of topologically complex three-dimensional microfluidic systems in PDMS by rapid prototyping,” Anal. Chem. 72:3158-3164, 2000). Methods for manufacture of nanoelectromechanical systems can be used (see, e.g., Craighead, Science 290:1532-36, 2000). Microfabricated chips are commercially available from sources such as Caliper Technologies Inc. (Mountain View, Calif.) and ACLARA BioSciences Inc. (Mountain View, Calif.).

Any materials known for use in integrated chips can be used in the disclosed apparatus, including silicon, silicon dioxide, silicon nitride, polydimethyl siloxane (PDMS), polymethylmethacrylate (PMMA), plastic, glass, quartz, etc. Part or all of the apparatus can be selected to be transparent to electromagnetic radiation at the excitation and emission frequencies used for Raman spectroscopy, such as glass, silicon, quartz or any other optically clear material. For fluid-filled compartments that can be exposed to nucleic acids and/or nucleotides, such as the reaction chamber, microfluidic channel, nanochannel or microchannel, the surfaces exposed to such molecules can be modified by coating, for example to transform a surface from a hydrophobic to a hydrophilic surface and/or to decrease adsorption of molecules to a surface. Surface modification of common chip materials such as glass, silicon and/or quartz is known in the art (e.g., U.S. Pat. No. 6,263,286). Such modifications can include, but are not limited to, coating with commercially available capillary coatings (Supelco, Bellafonte, Pa.), silanes with various functional groups such as polyethyleneoxide or acrylamide, or any other coating known in the art.

Nucleotides to be detected can be moved down a microfluidic channel, nanochannel or microchannel. A microchannel or nanochannel can have a diameter between about 3 nm and about 1 μm. The diameter of the channel can be selected to be slightly smaller in size than an excitatory laser beam. The channel can include a microcapillary (available, e.g., from ACLARA BioSciences Inc., Mountain View, Calif.) or a liquid integrated circuit (e.g., Caliper Technologies Inc., Mountain View, Calif.). Such microfluidic platforms require only nanoliter volumes of sample. Nucleotides can move down a microfluidic channel by bulk flow of solvent, by electro-osmosis or by any other technique known in the art.

Alternatively, microcapillary electrophoresis can be used to transport nucleotides. Microcapillary electrophoresis generally involves the use of a thin capillary or channel that can or can not be filled with a particular separation medium. Electrophoresis of appropriately charged molecular species, such as negatively charged nucleotides, occurs in response to an imposed electrical field. Although electrophoresis is often used for size separation of a mixture of components that are simultaneously added to a microcapillary, it can also be used to transport similarly sized nucleotides that are sequentially released from a nucleic acid molecule. Because the purine nucleotides are larger than the pyrimidine nucleotides and would therefore migrate more slowly, the length of the various channels and corresponding transit time past the detector can be kept to a minimum to prevent differential migration from mixing up the order of nucleotides. Alternatively, the separation medium filling the microcapillary can be selected so that the migration rates of purine and pyrimidine nucleotides are similar or identical. Methods of microcapillary electrophoresis have been disclosed, for example, by Woolley and Mathies (Proc. Natl. Acad. Sci. USA 91:11348-352, 1994).

Microfabrication of microfluidic devices, including microcapillary electrophoretic devices has been discussed in, e.g., Jacobsen et al. (Anal. Biochem, 209:278-283,1994); Effenhauser et al. (Anal. Chem. 66:2949-2953, 1994); Harrison et al. (Science 261:895-897, 1993) and U.S. Pat. No. 5,904,824. Typically, these methods include photolithographic etching of micron scale channels on silica, silicon or other crystalline substrates or chips, and can be readily adapted for use in the disclosed methods and apparatus. Smaller diameter channels, such as nanochannels, can be prepared by known methods, such as coating the inside of a microchannel to narrow the diameter, or using nanolithography, focused electron beam, focused ion beam or focused atom laser techniques.

Nanochannels can be made, for example, using a high-throughput electron-beam lithography system. Electron beam lithography can be used to write features as small as 5 nm on silicon chips. Sensitive resists, such as polymethyl-methacrylate, coated on silicon surfaces can be patterned without use of a mask. The electron beam array can combine a field emitter cluster with a microchannel amplifier to increase the stability of the electron beam, allowing operation at low currents. The SoftMask™ computer control system can be used to control electron beam lithography of nanoscale features on a silicon or other chip.

Alternatively, nanochannels can be produced using focused atom lasers. (e.g., Bloch et al., “Optics with an atom laser beam,” Phys. Rev. Lett. 87:123-321, 2001.) Focused atom lasers can be used for lithography, much like standard lasers or focused electron beams. Such techniques are capable of producing micron scale or even nanoscale structures on a chip. Dip-pen nanolithography can also be used to form nanochannels. (e.g., Ivanisevic et al., “‘Dip-Pen’ Nanolithography on Semiconductor Surfaces,” J. Am. Chem. Soc., 123: 7887-7889, 2001.) Dip-pen nanolithography uses atomic force microscopy to deposit molecules on surfaces, such as silicon chips. Features as small as 15 nm in size can be formed, with spatial resolution of 10 nm. Nanoscale channels can be formed by using dip-pen nanolithography in combination with regular photolithography techniques. For example, a micron scale line in a layer of resist can be formed by standard photolithography. Using dip-pen nanolithography, the width of the line (and the corresponding diameter of the channel after etching) can be narrowed by depositing additional resist compound on the edges of the resist. After etching of the thinner line, a nanoscale channel can be formed. Alternatively, atomic force microscopy can be used to remove photoresist to form nanometer scale features.

Ion-beam lithography can also be used to create nanochannels on a chip. (e.g., Siegel, “Ion Beam Lithography,” VLSI Electronics, Microstructure Science, Vol. 16, Einspruch and Watts eds., Academic Press, New York, 1987.) A finely focused ion beam can be used to directly write features, such as nanochannels, on a layer of resist without use of a mask. Alternatively, broad ion beams can be used in combination with masks to form features as small as 100 nm in scale. Chemical etching, for example with hydrofluoric acid, can be used to remove exposed silicon that is not protected by resist. The skilled artisan will realize that the techniques disclosed above are not limiting, and that nanochannels can be formed by any method known in the art.

In a non-limiting example, Borofloat glass wafers (Precision Glass & Optics, Santa Ana, Calif.) can be pre-etched for a short period in concentrated HF (hydrofluoric acid) and cleaned before deposition of an amorphous silicon sacrificial layer in a plasma-enhanced chemical vapor deposition (PECVD) system (PEII-A, Technics West, San Jose, Calif.). Wafers can be primed with hexamethyldisilazane (HMDS), spin-coated with photoresist (Shipley 1818, Marlborough, Mass.) and soft-baked. A contact mask aligner (Quintel Corp. San Jose, Calif.) can be used to expose the photoresist layer with one or more mask designs, and the exposed photoresist removed using a mixture of Microposit developer concentrate (Shipley) and water. Developed wafers can be hard-baked and the exposed amorphous silicon removed using CF4 (carbon tetrafluoride) plasma in a PECVD reactor. Wafers can be chemically etched with concentrated HF to produce the reaction chamber and any channels. The remaining photoresist can be stripped and the amorphous silicon removed.

Access holes can be drilled into the etched wafers with a diamond drill bit (Crystalite, Westerville, Ohio). A finished chip can be prepared by thermally bonding an etched and drilled plate to a flat wafer of the same size in a programmable vacuum furnace (Centurion VPM, J. M. Ney, Yucaipa, Calif.). Alternatively, the chip can be prepared by bonding two etched plates to each other. Alternative exemplary methods for fabrication of a reaction chamber chip are disclosed in U.S. Pat. Nos. 5,867,266 and 6,214,246.

In certain aspects, an apparatus according to the present invention includes a molecule dispenser. A molecular dispenser can be designed to release nucleotides into the reaction chamber. The molecule dispenser can release each type of nucleotide in equal amounts. A single molecule dispenser can be used to release all four nucleotides into the reaction chamber. Alternatively, the rate of release of the four types of nucleotides can be independently controlled, for example by using multiple molecule dispensers each releasing a single type of nucleotide. In certain methods, a single type of nucleotide can be released into the chamber at a time. Alternatively, all four types of nucleotides can be present in the reaction chamber simultaneously.

The molecular dispenser can be in the form of a pumping device. Pumping devices that can be used include a variety of micromachined pumps that are known in the art. For example, pumps having a bulging diaphragm, powered by a piezoelectric stack and two check valves are disclosed in U.S. Pat. Nos. 5,277,556, 5,271,724 and 5,171,132. Pumps powered by a thermopneumatic element are disclosed in U.S. Pat. No. 5,126,022. Piezoelectric peristaltic pumps using multiple membranes in series, or peristaltic pumps powered by an applied voltage are disclosed in U.S. Pat. No. 5,705,018. Published PCT Application No. WO 94/05414 discloses the use of a lamb-wave pump for transportation of fluid in micron scale channels. The skilled artisan will realize that the molecule dispenser is not limited to the pumps disclosed herein, but can incorporate any design for the measured disbursement of very low volume fluids known in the art.

The molecular dispenser can take the form of an electrohydrodynamic pump (e.g., Richter et al., Sensors and Actuators 29:159-165 1991; U.S. Pat. No. 5,126,022). Typically, such pumps employ a series of electrodes disposed across one surface of a channel or reaction/pumping chamber. Application of an electric field across the electrodes results in electrophoretic movement of charged species in the sample. Indium-tin oxide films can be particularly suited for patterning electrodes on substrate surfaces, for example a glass or silicon substrate. These methods can also be used to draw nucleotides into the reaction chamber. For example, electrodes can be patterned on the surface of the molecule dispenser and modified with suitable functional groups for coupling nucleotides to the surface of the electrodes. Application of a current between the electrodes on the surface of the molecule dispenser and an opposing electrode results in electrophoretic movement of the nucleotides into the reaction chamber.

A detection unit can be designed to detect and/or quantify nucleotides by Raman spectroscopy. Various methods for detection of nucleotides by Raman spectroscopy are known in the art (see, e.g., U.S. Pat. Nos. 5,306,403; 6,002,471; 6,174,677). Variations on surface enhanced Raman spectroscopy (SERS) or surface enhanced resonance Raman spectroscopy (SERRS) have been disclosed. In SERS and SERRS, the sensitivity of the Raman detection is enhanced by a factor of 10⁶ or more for molecules adsorbed on roughened metal surfaces, such as silver, gold, platinum, copper or aluminum surfaces, or on nanostructured surfaces.

A non-limiting example of a detection unit is disclosed in U.S. Pat. No. 6,002,471. In this embodiment, the excitation beam is generated by either a Nd:YAG laser at 532 nm wavelength or a frequency doubled Ti:sapphire laser at 365 nm wavelength. However, the excitation wavelength can vary considerably, without limiting the methods of the present invention. For example, as illustrated in the examples herein, in certain aspects, the excitation beam is delivered at a wavelength between about 750 to about 950 nm. Pulsed laser beams or continuous laser beams can be used. The excitation beam passes through confocal optics and a microscope objective, and is focused onto the reaction chamber. The Raman emission light from the nucleotides is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector. The detector includes an avalanche photodiode interfaced with a computer for counting and digitization of the signal. In certain embodiments, a mesh including silver, gold, platinum, copper or aluminum can be included in the reaction chamber or channel to provide an increased signal due to surface enhanced Raman or surface enhanced Raman resonance. Alternatively, nanoparticles that include a Raman-active metal can be included.

Alternative embodiments of detection units are disclosed, for example, in U.S. Pat. No. 5,306,403, including a Spex Model 1403 double-grating spectrophotometer equipped with a gallium-arsenide photomultiplier tube (RCA Model C31034 or Burle Industries Model C3103402) operated in the single-photon counting mode. The excitation source is a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm (U.S. Pat. No. 6,174,677). The excitation beam can be spectrally purified with a bandpass filter (Corion) and can be focused on the reaction chamber using a 6× objective lens (Newport, Model L6X). The objective lens can be used to both excite the nucleotides and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) can be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors can be used, such as charged injection devices, photodiode arrays or phototransistor arrays.

Any suitable form or configuration of Raman spectroscopy or related techniques known in the art can be used for detection of nucleotides, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

In embodiments of the present invention, depending on the specific nucleotide, nucleoside, or base being detected, different numbers of molecules can be detected. Typically, smaller numbers of molecules can be detected for purines as opposed to pyrimidines and for bases versus nucleotides. For example, where the nucleotide, nucleoside, or base includes adenine, 10 or less, 5 or less, or 1 molecule of the nucleotide, nucleoside, or base can be detected.

In examples where the nucleotide, nucleoside, or base includes guanine, for example, between about 50 and about 100 molecules, for example about 60 molecules of a guanine base are detected. In examples where the nucleotide, nucleoside, or base includes cytosine between about 1000 and 10000 molecules, for example 5000 and 7000 can be detected. In examples where the nucleotide, nucleoside, or base includes thymine, between about 1000 and 10000, more specifically, for example, between about 5000 to about 7000 molecules can be detected.

The following examples are intended to illustrate but not limit the invention.

### EXAMPLE 1

### Raman Detection of Nucleotides

Methods and Apparatus

In a non-limiting example, the excitation beam of a Raman detection unit was generated by a titanium:sapphire laser (Mira by Coherent) at a near-infrared wavelength (750˜950 nm) or a gallium aluminum arsenide diode laser (PI-ECL series by Process Instruments) at 785 nm or 830 nm. Pulsed laser beams or continuous beams were used. The excitation beam was passed through a dichroic mirror (holographic notch filter by Kaiser Optical or a dichromatic interference filter by Chroma or Omega Optical) into a collinear geometry with the collected beam. The transmitted beam passed through a microscope objective (Nikon LU series), and was focused onto the Raman active substrate where target analytes (nucleotides or purine or pyrimidine bases) were located.

The Raman scattered light from the analytes was collected by the same microscope objective, and passed the dichroic mirror to the Raman detector. The Raman detector included a focusing lens, a spectrograph, and an array detector. The focusing lens focused the Raman scattered light through the entrance slit of the spectrograph. The spectrograph (Acton Research) included a grating that dispersed the light by its wavelength. The dispersed light was imaged onto an array detector (back-illuminated deep-depletion CCD camera by Roperscientific). The array detector was connected to a controller circuit, which was connected to a computer for data transfer and control of the detector function.

For surface-enhanced Raman spectroscopy (SERS), the Raman active substrate consisted of metallic nanoparticles or metal-coated nanostructures. Silver nanoparticles, ranging in size from 5 to 200 nm, were made by the method of Lee and Meisel (J. Phys. Chem., 86:3391, 1982). Alternatively, samples were placed on an aluminum substrate under the microscope objective. The Figures discussed below were collected in a stationary sample on the aluminum substrate. The number of molecules detected was determined by the optical collection volume of the illuminated sample. Detection sensitivity down to the single molecule level was demonstrated.

Single nucleotides can also be detected by SERS using a 100 μm or 200 μm microfluidic channel. Nucleotides can be delivered to a Raman active substrate through a microfluidic channel (between about 5 and 200 μm wide). Microfluidic channels can be made by molding polydimethylsiloxane (PDMS), using the technique disclosed in Anderson et al. (“Fabrication of topologically complex three-dimensional microfluidic systems in PDMS by rapid phototyping,” Anal. Chem. 72:3158-3164, 2000).

Where SERS was performed in the presence of silver nanoparticles, the nucleotide, purine or pyrimidine analyte was mixed with LiCl (90 μM final concentration) and nanoparticles (0.25 M final concentration silver atoms). SERS data were collected using room temperature analyte solutions.

Nucleoside monophosphates, purine bases and pyrimidine bases were analyzed by SERS, using the system disclosed above. Table 1 shows the present detection limits for various analytes of interest.

TABLE 1
SERS Detection of Nucleoside Monophosphates, Purines and Pyrimidines

| | | Number of Molecules |
|---|---|---|
| Analyte | Final Concentration | Detected |
| dAMP | 9 picomolar (pM) | ˜1 molecule |
| Adenine | 9 pM | ˜1 molecule |
| dGMP | 90 μM | 6 × 10⁶ |
| Guanine | 909 pM | 60 |
| dCMP | 909 μM | 6 × 10⁷ |
| Cytosine | 90 nM | 6 × 10³ |
| dTMP | 90 μM | 6 × 10⁶ |
| Thymine | 90 nM | 6 × 10³ |

Conditions were optimized for adenine nucleotides only. LiCL (90 μM final concentration) was determined to provide optimal SERS detection of adenine nucleotides. Detection of other nucleotides can be facilitated by use of other alkali-metal halide salts, such as NaCl, KCl, RbCl or CsCl. The claimed methods are not limited by the electrolyte solution used, and it is contemplated that other types of electrolyte solutions, such as MgCl₂, CaCl₂, NaF, KBr, LiI, etc. can be of use. The skilled artisan will realize that electrolyte solutions that do not exhibit strong Raman signals will provide minimal interference with SERS detection of nucleotides. The results demonstrate that the Raman detection system and methods disclosed above were capable of detecting and identifying single molecules of nucleotides and purine bases. This is the first report of Raman detection of unlabeled nucleotides at the single nucleotide level.

### EXAMPLE 2

### Raman Emission Spectra of Nucleotides, Purines and Pyrimidines

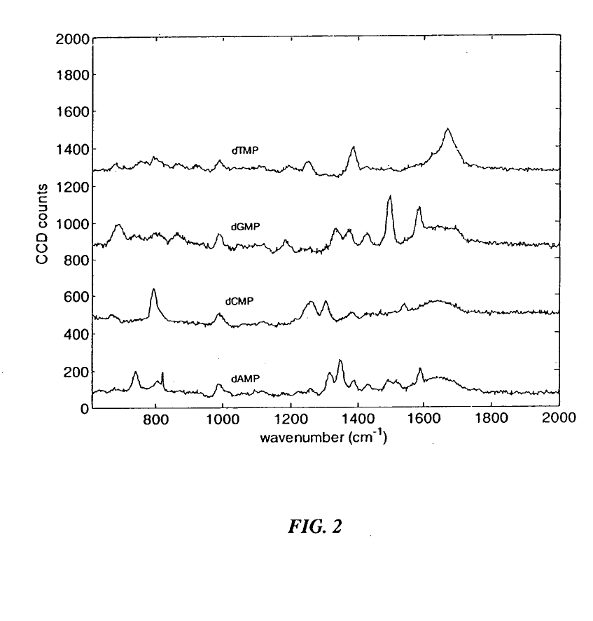

The Raman emission spectra of various analytes of interest were obtained using the protocol of Example 1, with the indicated modifications. FIG. 2 shows the Raman emission spectra of a 100 mM solution of each of the four nucleoside monophosphates, in the absence of surface enhancement and without Raman labels. No LiCl was added to the solution. A 10 second data collection time was used. Excitation occurred at 514 nm. Lower concentrations of nucleotides can be detected with longer collection times, added electrolytes and/or surface enhancement. For each of the following figures, a 785 nm excitation wavelength was used. As shown in FIG. 2, the unenhanced Raman spectra showed characteristic emission peaks for each of the four unlabeled nucleoside monophosphates.

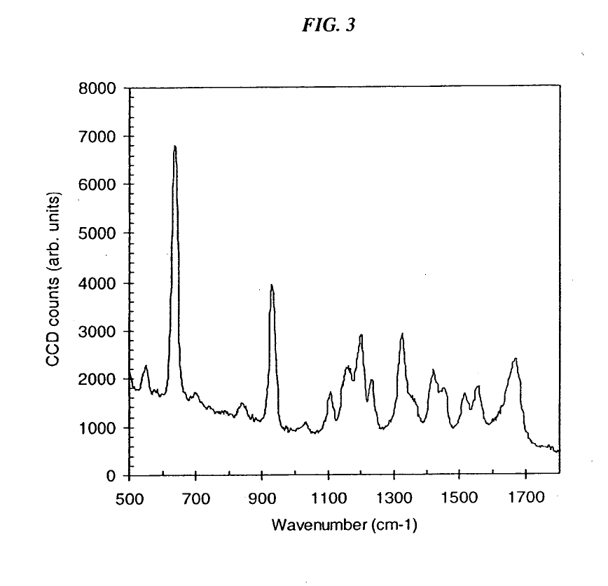

FIG. 3 shows the SERS spectrum of a 1 nm solution of guanine, in the presence of LiCl and silver nanoparticles. Guanine was obtained from dGMP by acid treatment, as discussed in Nucleic Acid Chemistry, Part 1, L. B. Townsend and R. S. Tipson (eds.), Wiley-Interscience, New York, 1978. The SERS spectrum was obtained using a 100 msec data collection time.

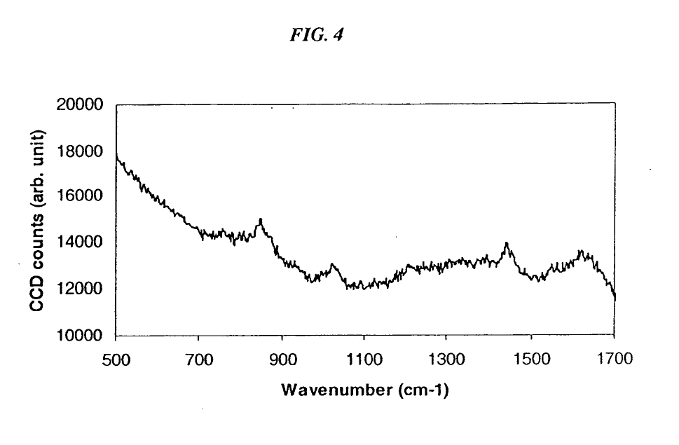

FIG. 4 shows the SERS spectrum of a 100 nM cytosine solution. Data were collected using a 1 second collection time.

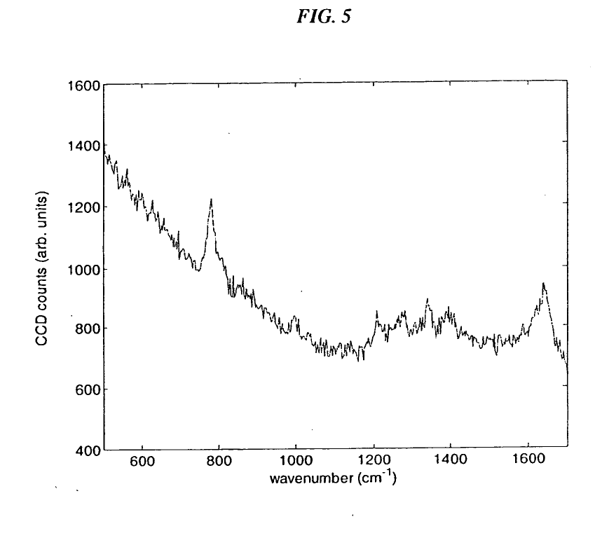

FIG. 5 shows the SERS spectrum of a 100 nM thymine solution. Data were collected using a 100 msec collection time.

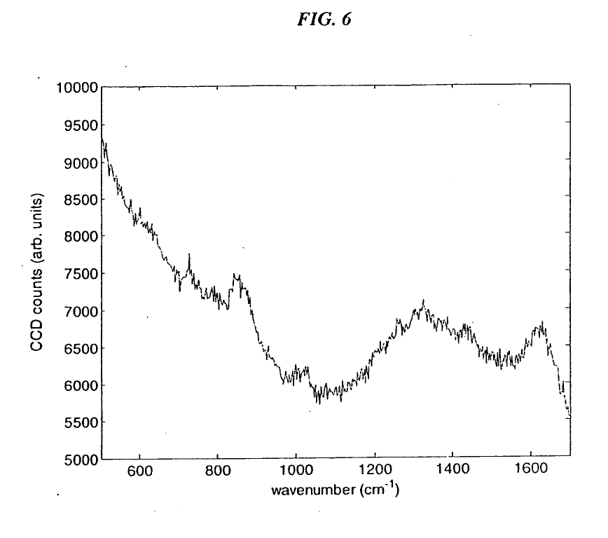

FIG. 6 shows the SERS spectrum of a 100 pM adenine solution. Data were collected for 1 second.

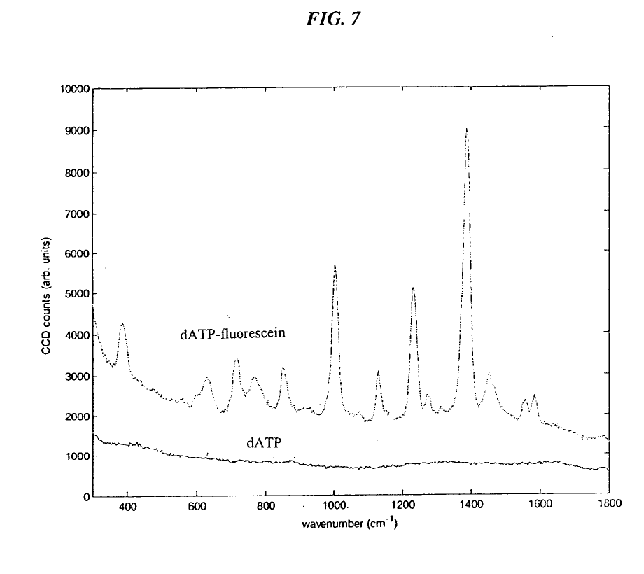

FIG. 7 shows the SERS spectrum of a 500 nM solution of dATP (lower trace) and fluorescein-labeled dATP (upper trace). dATP-fluorescein was purchased from Roche Applied Science (Indianapolis, Ind.). The Figure shows a strong increase in SERS signal due to labeling with fluorescein. Data was collected for 100 msec.

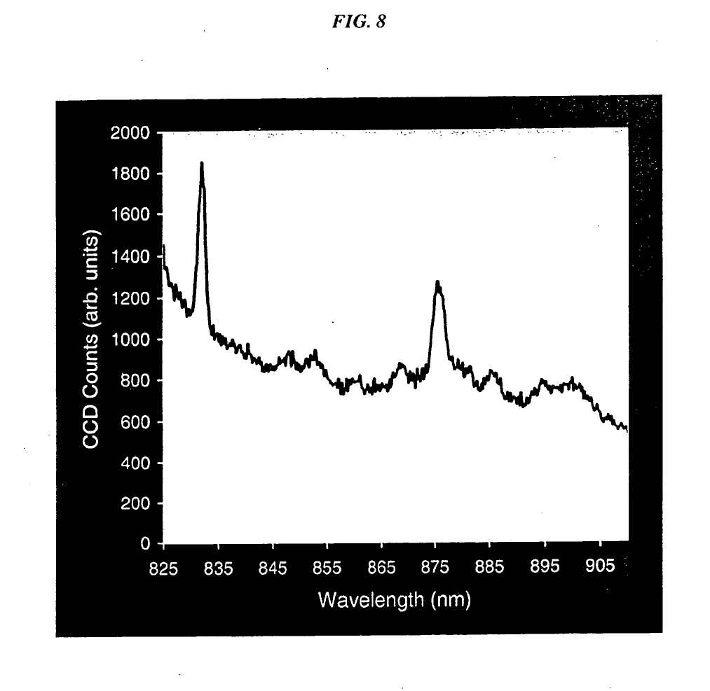

FIG. 8 shows the SERS of a 0.9 nM solution of adenine. The detection volume was 100 to 150 femtoliters, containing an estimated 60 molecules of adenine. Data was collected for 100 msec.

### EXAMPLE 3

### SERS Detection of Nucleotides and Amplification Products

Silver Nanoparticle Formation

Silver nanoparticles used for SERS detection were produced according to Lee and Meisel (1982). Eighteen milligrams of AgNO³ were dissolved in 100 mL (milliliters) of distilled water and heated to boiling. Ten mL of a 1% sodium citrate solution was added drop-wise to the AgNO³ solution over a 10 min period. The solution was kept boiling for another hour. The resulting silver colloid solution was cooled and stored.

SERS Detection of Adenine

The Raman detection system was as disclosed in Example 1. One mL of silver colloid solution was diluted with 2 mL of distilled water. The diluted silver colloid solution (160 μL) (microliters) was mixed with 20 μL of a 10 nM (nanomolar) adenine solution and 40 μL of LiCl (0.5 molar) on an aluminum tray. The LiCl acted as a Raman enhancing agent for adenine. The final concentration of adenine in the sample was 0.9 nM, in a detection volume of about 100 to 150 femtoliters, containing an estimated 60 molecules of adenine. The Raman emission spectrum was collected using an excitation source at 785 nm excitation, with a 100 millisecond collection time. As shown in FIG. 8, this procedure demonstrated the detection of 60 molecules of adenine, with strong emission peaks detected at about 833 nm and 877 nm. As discussed in Example 1, single molecule detection of adenine has been shown using the disclosed methods and apparatus.

Rolling Circle Amplification

One picomole (pmol) of a rolling circle amplification (RCA) primer was added to 0.1 pmol of circular, single-stranded M13 DNA template. The mixture was incubated with 1×T7 polymerase 160 buffer (20 mM (millimolar) Tris-HCl, pH 7.5, 10 mM MgCl₂, 1 mM dithiothreitol), 0.5 mM dNTPs and 2.5 units of T7 DNA polymerase for 2 hours at 37° C., resulting in formation of an RCA product. A negative control was prepared by mixing and incubating the same reagents without the DNA polymerase.

SERS Detection of RCA Product

One μL of the RCA product and 1 μL of the negative control sample were separately spotted on an aluminum tray and air-dried. Each spot was rinsed with 5 μL of IX PBS (phosphate buffered saline). The rinse was repeated three times and the aluminum tray was air-dried after the final rinse.

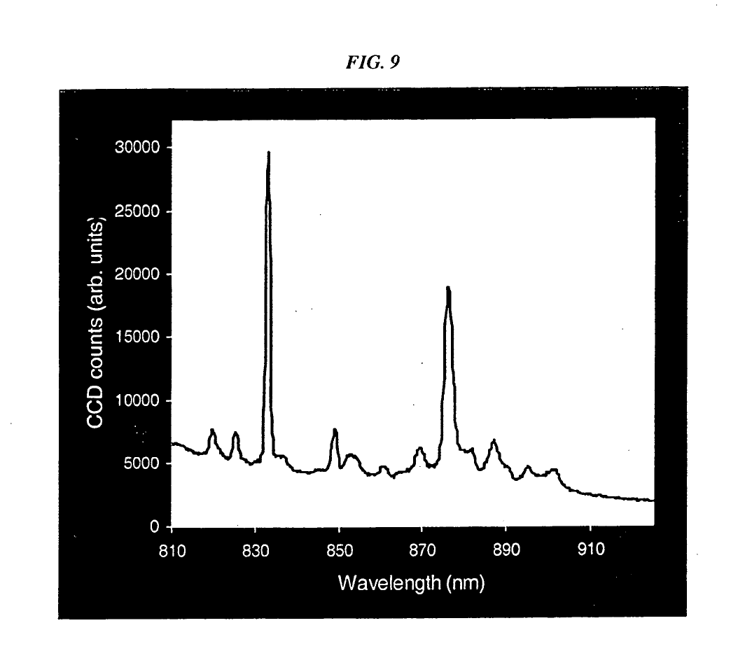

One milliliter of silver colloid solution prepared as above was diluted with 2 mL of distilled water Eight microliters of the diluted silver colloid solution was mixed with 2 μL of 0.5 M LiCl and added to the RCA product spot on the aluminum tray. The same solution was added to the negative control spot. The Raman signals were collected as disclosed above. As demonstrated in FIG. 9, an RCA product was detectable by SERS, with emission peaks at about 833 and 877 nm. Under the conditions of this protocol, with an LiCl enhancer, the signal strength from the adenine moieties is stronger than those for guanine, cytosine and thymine. The negative control (not shown) showed that the Raman signal was specific for the RCA product, as no signal was observed in the absence of amplification.

Nucleic Acid Sequencing

Human chromosomal DNA is purified according to Sambrook et al. (1989). Following digestion with Bam HI, the genomic DNA fragments are inserted into the multiple cloning site of the pBluescript® I1 phagemid vector (Stratagene, Inc., La Jolla, Calif.) and replicated in E. coli. After plating on ampicillin-containing agarose plates a single colony is selected and grown up for sequencing. Single-stranded DNA copies of the genomic DNA insert are rescued by co-infection with helper phage. After digestion in a solution of proteinase K:sodium dodecyl sulphate (SDS), the DNA is phenol extracted and then precipitated by addition of sodium acetate (pH 6.5, about 0.3 M) and 0.8 volumes of 2-propanol. The DNA containing pellet is resuspended in Tris-EDTA buffer and stored at −20° C. until use. Agarose gel electrophoresis shows a single band of purified DNA.

M13 forward primers complementary to the known pBluescript® sequence, located next to the genomic DNA insert, are purchased from Midland Certified Reagent Company (Midland, Tex.). The primers are covalently modified to contain a biotin moiety attached to the 5′ end of the oligonucleotide. The biotin group is covalently linked to the 5′-phosphate of the primer via a (CH₂)₆ spacer. Biotin-labeled primers are allowed to hybridize to the ssDNA template molecules prepared from the pBluescript® vector. The primer-template complexes are then attached to streptavidin-coated beads according to Dorre et al. (Bioimaging 5: 139-152, 1997). At appropriate DNA dilutions, a single primer-template complex is attached to a single bead. A bead containing a single primer-template complex is inserted into the reaction chamber of a sequencing apparatus.

The primer-template is incubated with modified T7 DNA polymerase (United States Biochemical Corp., Cleveland, Ohio). The polymerase is confined to the reaction chamber by optical trapping (Goodwin et al., 1996, Acc. Chem. Res. 29:607-619). The reaction mixture contains unlabled deoxyadenosine-5′-triphosphate (dATP) and deoxyguanosine-5′-triphosphate (dGTP), digoxigenin-labeled deoxyuridine-5′-triphosphate (digoxigenin-dUTP) and rhodamine-labeled deoxycytidine-5′-triphosphate (rhodamine-dCTP). The polymerization reaction is allowed to proceed at 37° C.

A continuous flow of all four nucleotides is channeled through the reaction chamber. Nucleotide concentration is measured before and after the reaction chamber by SERS. The incorporation of nucleotides into the complementary strand is determined by a decrease in concentration of nucleotide exiting the reaction chamber. The time-dependent uptake of nucleotides is used to derive the sequence of the template strand.

In an alternative method, only a single type of nucleotide is provided to the reaction chamber at one time. Each of the four types of nucleotide is sequentially added to the reaction chamber. The amount of nucleotide provided is proportional to the amount of template nucleic acid in the reaction chamber. When a nucleotide is complementary to the next base in the template strand, a large depletion in nucleotide concentration is observed in the flow-through channel exiting the reaction chamber. When any of the other three types of nucleotides is added, little change in nucleotide concentration is observed. The process is repeated for each base in the template strand to determine the nucleic acid sequence.

Although the invention has been described with reference to the above examples, it will be understood that modifications and variations are encompassed within the spirit and scope of the invention. Accordingly, the invention is limited only by the following claims.

1. A method to detect a nucleotide, nucleoside, or base, comprising:
a) depositing the nucleotide, nucleoside, or base on a substrate comprising aluminum or comprising a metal-coated nanostructure;
b) irradiating the deposited nucleotide, nucleoside, or base; and
c) detecting Raman spectra from the irradiated nucleotide, nucleoside, or base, thereby detecting the nucleotide, nucleoside, or base.

2. The method of claim 1, wherein the nucleotide, nucleoside, or base is deposited on one or more silver nanoparticles between about 5 and 200 nm in diameter, before being detected.

3. The method of claim 2, wherein the nucleotide, nucleoside, or base is contacted with an alkali-metal halide salt before being detected.

4. The method of claim 3, wherein the alkali-metal halide salt is lithium chloride.

5. The method of claim 4, wherein the nucleotide, nucleoside, or base comprises adenine.

6. The method of claim 5, wherein the lithium chloride is used at a concentration of about 50 to about 150 micromolar.

7. The method of claim 5, wherein the lithium chloride is used at a concentration of about 90 micromolar.

8. The method of claim 7, wherein 10 or less molecules of a nucleotide, nucleoside, or base comprising adenine are detected.

9. The method of claim 7, wherein 1 molecule of a nucleotide, nucleoside, or base comprising adenine is detected.

10. The method of claim 4, wherein the nucleotide, nucleoside, or base comprises guanine.

11. The method of claim 10, wherein between about 50 and about 100 molecules of a guanine base are detected.

12. The method of claim 4, wherein the nucleotide, nucleoside, or base comprises cytosine.

13. The method of claim 12, wherein between about 1000 and 10000 molecules of a cytosine base are detected.

14. The method of claim 4, wherein the nucleotide, nucleoside, or base comprises thymine.

15. The method of claim 14, wherein between about 1000 and 10000 molecules of a thymine base are detected.

16. The method of claim 1, wherein the nucleotide, nucleoside, or base are associated with a Raman label.

17. The method of claim 1, wherein a base is detected.

18. An apparatus comprising:
a) a reaction chamber containing a single template nucleic acid molecule attached to an immobilization surface;
b) a channel in fluid communication with the reaction chamber; and
c) a Raman detection unit operably coupled to the channel.

19. The apparatus of claim 18, wherein the Raman detection unit is capable of detecting at least one nucleotide at the single molecule level.

20. The apparatus of claim 18, wherein the concentrations of nucleotides is measured by Raman spectroscopy as they flow through the channel.

21. The apparatus of claim 18, further comprising metal nanoparticles in the channel.

22. The apparatus of claim 18, wherein the channel diameter is between about 100 and about 200 micrometers in diameter.

23. The apparatus of claim 18, further comprising a silver, gold, platinum, copper or aluminum mesh inside the channel.

24. A method to determine a nucleotide occurrence at a target position of one or more template nucleic acid molecules, comprising:
a) contacting the one or more template nucleic acid molecules with a reaction mixture comprising a primer, a polymerase, and an initial concentration of a first nucleotide, wherein the 3′ nucleotide of the primer binds to the template nucleic acid adjacent to the target nucleotide position to form a post-reaction mixture; and
b) determining the concentration of the first nucleotide in the post-reaction mixture using Raman spectroscopy, wherein a decrease in the post-reaction concentration of the first nucleotide identifies an extension reaction product, thereby identifying the nucleotide occurrence at the target position; and
c) repeating steps a-b with a different nucleotide until the nucleotide occurrence is identified.

25. The method of claim 24, wherein the nucleotide is attached to a Raman label before it is detected by Raman spectroscopy.

26. The method of claim 24, wherein the nucleotide is attached to a fluorophore before it is detected by Raman spectroscopy.

27. The method of claim 24, wherein the one or more template nucleic acid molecules are isolated from a biological sample before being contacted with the first reaction mixture.

28. The method of claim 24, wherein the concentration of a purine base is detected.

29. A method to sequence one or more nucleic acid molecules, comprising:
a) contacting the one or more template nucleic acid molecules with nucleotides, a primer, and a polymerase to form a reaction mixture, the one or more template nucleic acid molecules or the primer being immobilized on a solid support;
b) synthesizing one or more complementary strands to the one or more template nucleic acid molecules;
d) measuring the concentrations of the nucleotides in the reaction mixture by Raman spectroscopy; and
e) determining the sequence of the template nucleic acid from the nucleotides incorporated into the complementary strand.

30. The method of claim 29, further comprising separating the nucleotides from the template nucleic acid molecule before the nucleotide concentrations are measured.

31. The method of claim 29, wherein a single type of nucleotide is exposed to the template at one time.

32. The method of claim 29, wherein all four types of nucleotides are exposed to the template simultaneously.

33. The method of claim 29, wherein Raman labels are attached to each nucleotide.

34. The method of claim 29, wherein Raman labels are attached to the pyrimidine nucleotides.

35. The method of claim 29, wherein the nucleotide concentrations are measured by surface enhanced Raman scattering, surface enhanced resonance Raman scattering, stimulated Raman scattering, inverse Raman, stimulated gain Raman spectroscopy, hyper-Raman scattering or coherent anti-Stokes Raman scattering.
